(19) United States
(12) Patent Application Publication
(10) Pub. No.: US 2025/0017105 A1
(43) Pub. Date: Jan. 9, 2025

(54) FUSED RING ACCEPTOR MATERIAL AND METHOD OF MANUFACTURING THE SAME

(71) Applicant: National Yang Ming Chiao Tung University, Hsinchu City (TW)

(72) Inventors: Yen-Ju CHENG, Taipei City (TW); Yung-Jing XUE, Chiayi County (TW); Han-Cheng LU, Yunlin County (TW); Jun-Cheng HONG, Taipei City (TW); Kuo-Hsiu HUANG, Taichung City (TW)

(21) Appl. No.: 18/757,513

(22) Filed: Jun. 28, 2024

(30) Foreign Application Priority Data
Jun. 28, 2023 (TW) 112124164

(51) Int. Cl. H10K 85/60; C07D 495/22; H10K 30/20

(52) U.S. Cl. CPC H10K 85/657; C07D 495/22; H10K 85/626; H10K 30/20

(57) ABSTRACT

A fused ring acceptor material includes a structure of following formula (I).

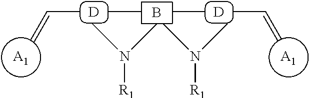

formula (I), where R₁ is a C₁-C₂₄ alkyl, a phenyl, a phenyl derivative, a furyl, a furyl derivative, a thienyl, a thienyl derivative, a selenophene or a selenophene derivative; B is

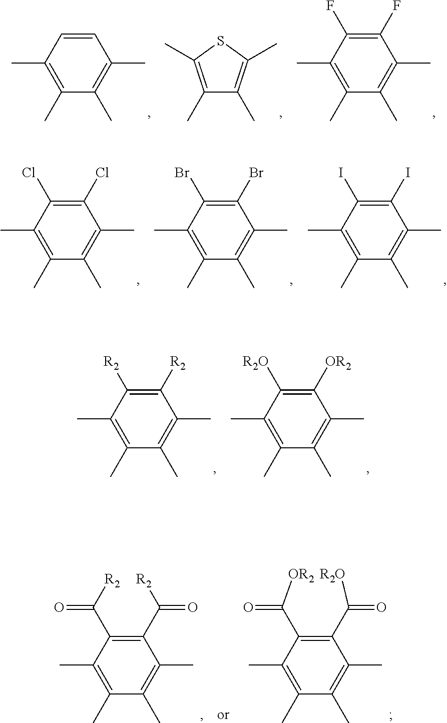

R₂ is a C₁-C₂₄ alkyl, a phenyl, a phenyl derivative, a thienyl or a thienyl derivative; D is

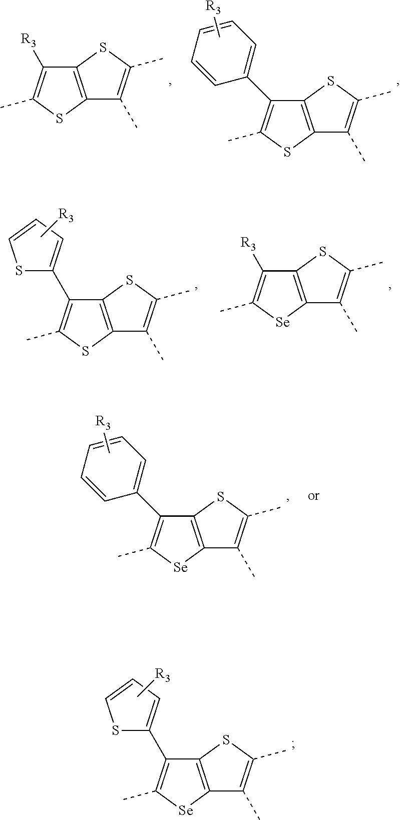

R₃ is a hydrogen atom, a halogen, a C₁-C₂₄ alkyl or a C₁-C₂₄ alkoxy; and A₁ is

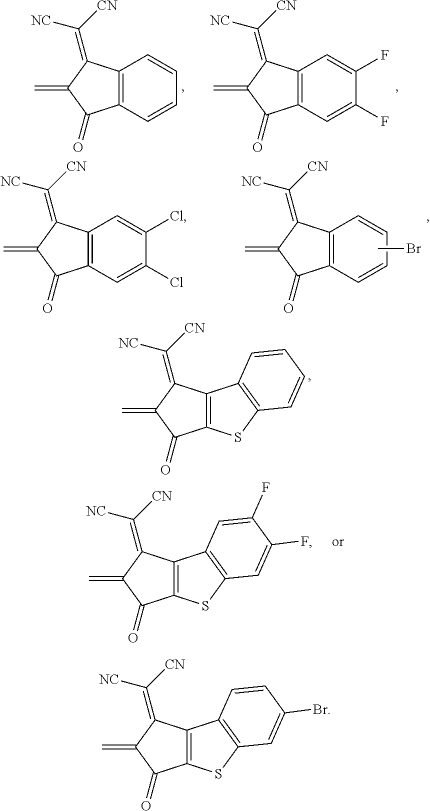

This application claims priority to Taiwan Application Serial Number 112124164, filed Jun. 28, 2023, which is herein incorporated by reference.

### BACKGROUND

### Field of Invention

The present disclosure relates to a fused ring acceptor material and a method of manufacturing the fused ring acceptor material.

### Description of Related Art

With the evolution of the times, the consumption of energy resources such as coal, oil, natural gas and nuclear energy is increasing day by day, and the energy crisis has also emerged. Solar power generation is a renewable and environmentally friendly way of generating electricity that can reduce environmental pollution. The first generation of solar cells is mainly silicon based, which has high power conversion efficiency (PCE). The second generation is CdTe thin-film solar cells, but the toxicity of its raw materials and the production process cause great pollution to the environment. Therefore, the third generation of organic solar cells was born, which includes dye-sensitized solar cells (DSSC), nanocrystalline cells and organic photovoltaics (OPV).

The development of the organic solar cells has entered the era of non-fullerene acceptors (NFA) after 2015. The fullerene acceptor that has been traditionally used for a long time in the past is replaced, and thus efficiency is greatly improved. The current high-efficiency organic non-fullerene acceptor material is called TPBT-4F((2,20-((2Z,20Z)-((12,13-bis(2-ethylhexyl)-3,9-diundecyl-12,13-dihydro-[1,2,5]thiadiazolo[3,4-e]thieno[2,″30′:4′,50]thieno[20,30:4,5]pyrrolo[3,2-g]thieno[20,30:4,5]thieno[3,2-b]indole-2,10-diyl)bis(methanylylidene))bis(5,6-difluoro-3-oxo-2,3-dihydro-1H-indene-2,1-diylidene))dimalononitrile)). Its basic structure may be expressed as A₁-D^{N}A₂^{N}D-A₁, and A₁ is a terminal acceptor with an electron-withdrawing group, 2-(5,6-difluoro-3-oxo-2,3-dihydro-1H-inden-1-ylidene)malononitrile (FIC), and central D^{N}A₂^{N}D is a ladder-shaped conjugated system with a fused ring, where A₂ is benzothiadiazole (BT) with ability to pull electrons, and D is thienothiophene (TT) unit, and D and A₂ are bridged by a nitrogen atom to form the ladder-shaped structure of the fused ring. A light absorption range of BTPIC can reach 700 nm to 950 nm, and it has good solubility after introducing a side chain group, so it can be processed in a wet process. Such derivatives can achieve photoelectric conversion efficiency of 15% to 18% when paired with a suitable donor, such as P-type conjugated polymer (PM6). On the basis of this structure, both side chain group modification and terminal group modification can achieve good efficiency. It is the material with the greatest commercial development potential today.

However, it is still relatively difficult to synthesize this type structure of A₁-D^{N}A₂^{N}D-A₁, and it is very difficult to mass-produce it. For example, in the process of synthesizing D^{N}A₂^{N}D, a step of cadogan cyclization of a nitro group is required to form a pyrrole unit. This step requires the use of an organophosphorus reagent and must be carried out at a high temperature of 180° C., and the yield is only 20-30%. Regarding the selection of the central molecule A₂, since the central molecule for the cyclization reaction must have an electron-withdrawing substituent to proceed, diversity of molecular structures of this type of the non-fullerene acceptor is also limited. These are still problems that still need to be overcome in previous research and technology.

### SUMMARY

Regarding problems of difficulty in preparing organic non-fullerene acceptor materials in existing organic solar cells, one aspect of the present disclosure provides a fused ring acceptor material, which includes a structure of following formula (I).

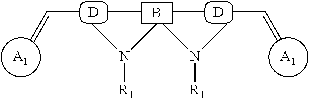

formula (I), where R₁ is a C₁-C₂₄ alkyl, a phenyl, a phenyl derivative, a furyl, a furyl derivative, a thienyl, a thienyl derivative, a selenophene or a selenophene derivative; B is

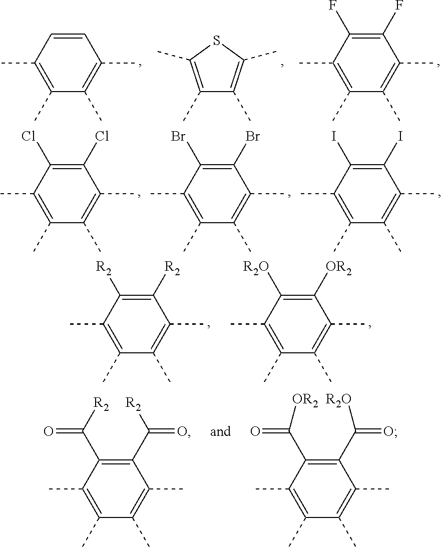

R₂ is a C₁-C₂₄ alkyl, a phenyl, a phenyl derivative, a thienyl or a thienyl derivative; D is

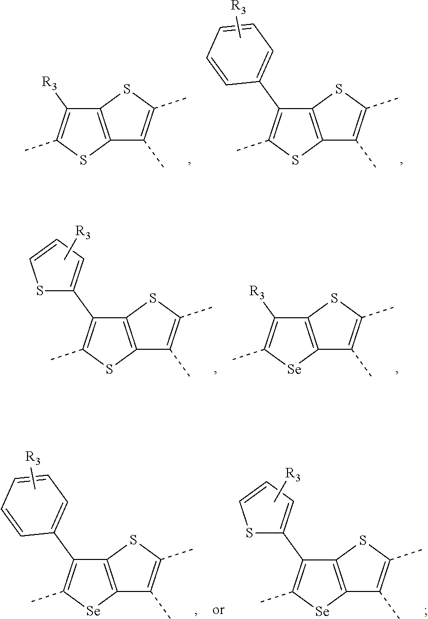

R₃ is a hydrogen atom, a halogen, a C₁-C₂₄ alkyl or a C₁-C₂₄ alkoxy; and A₁ is

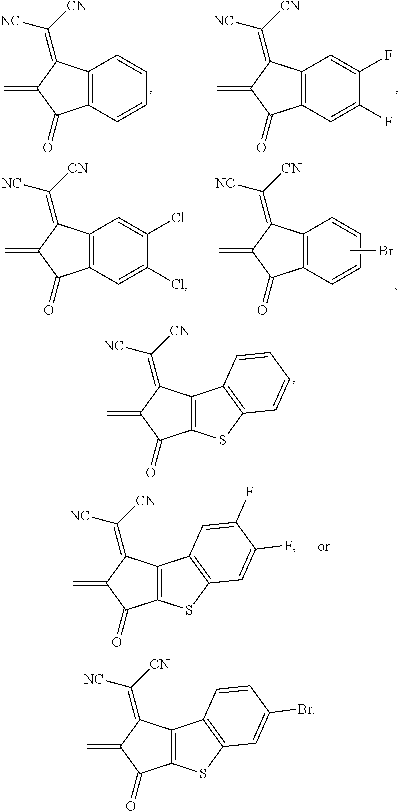

According to some embodiments of the present disclosure, when R₂ is the phenyl derivative, the phenyl derivative includes at least one group of a C₁-C₂₄ alkyl, a C₁-C₂₄ alkoxy, a halogen or a combination thereof.

According to some embodiments of the present disclosure, when R₂ is the thienyl derivative, the thienyl derivative includes at least one group of a C₁-C₂₄ alkyl, a C₁-C₂₄ alkoxy, a halogen or a combination thereof.

Another aspect of the present disclosure provides a method of manufacturing a fused ring acceptor material, which includes following steps. A coupling reaction is performed between a compound 1 having dibromothienothiophene and a halogen-containing aromatic ring to obtain a compound 2.

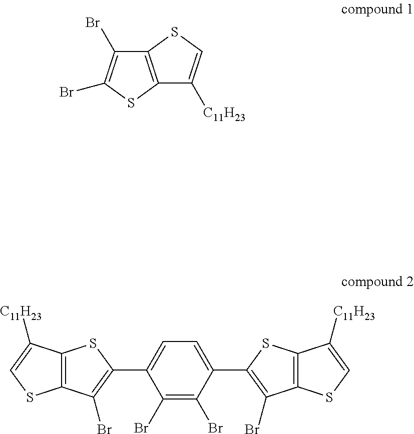

A Buchwald-Hartwig amination reaction is performed between the compound 2 and alkylamine to obtain a compound 3.

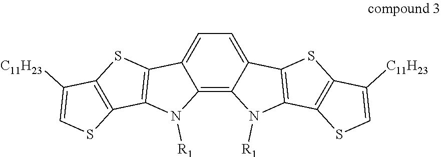

A Vilsmeier-Haack reaction is performed on the compound 3 to obtain a compound 4.

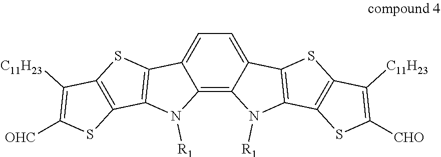

A condensation reaction is performed between the compound 4 and an A₁ group-containing acceptor to obtain a fused ring non-fullerene acceptor material, where R₁ is a C₁-C₂₄ alkyl, a phenyl, a phenyl derivative, a furyl, a furyl derivative, a thienyl, a thienyl derivative, a selenophene or a selenophene derivative; and the A₁ group-containing acceptor includes

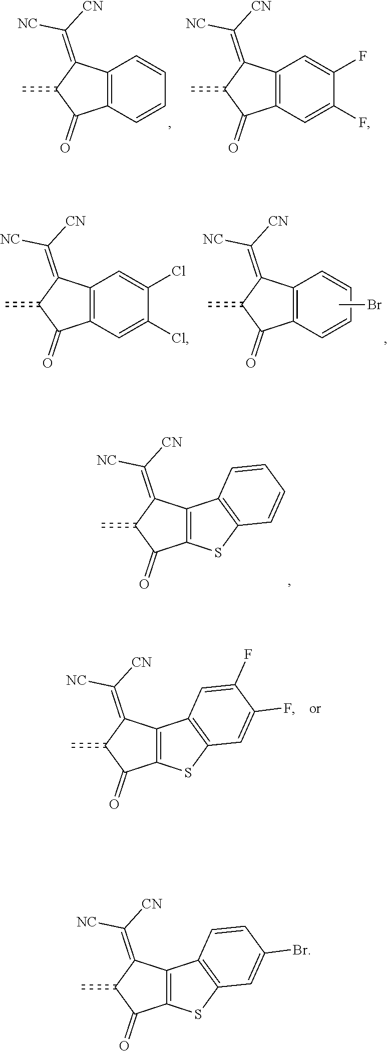

According to some embodiments of the present disclosure, forming the compound 1 having dibromothienothiophene includes following steps. A Friedel-crafts reaction is performed between 3-bromothiophene and acyl chloride to obtain a compound 5.

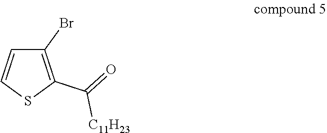

A cyclization reaction is performed between the compound 5 and thiol to obtain a compound 6.

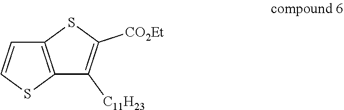

A bromination reaction is performed on the compound 6 to obtain a compound 7.

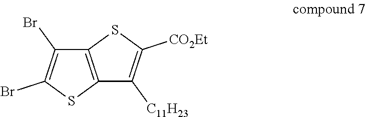

The compound 7 is hydrolyzed to obtain a compound 8.

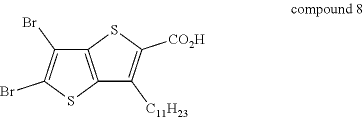

A decarboxylation reaction is performed on the compound 8 to obtain the compound 1 having dibromothienothiophene.

According to some embodiments of the present disclosure, the fused ring non-fullerene acceptor material has a structure of following formula (I):

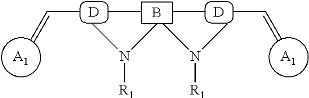

formula (I), where R₁ is a C₁-C₂₄ alkyl, a phenyl, a phenyl derivative, a furyl, a furyl derivative, a thienyl, a thienyl derivative, a selenophene or a selenophene derivative; B is

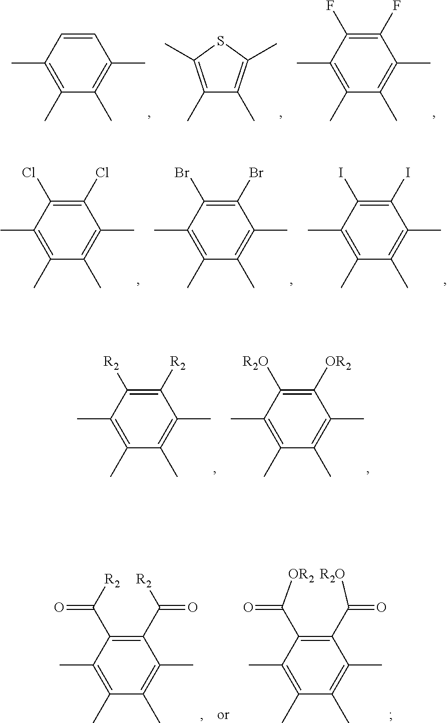

R₂ is a C₁-C₂₄ alkyl, a phenyl, a phenyl derivative, a thienyl or a thienyl derivative; D is

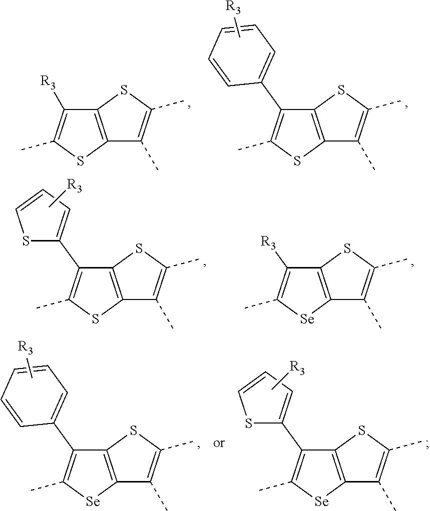

R₃ is a hydrogen atom, a halogen, a C₁-C₂₄ alkyl or a C₁-C₂₄ alkoxy; and A₁ is

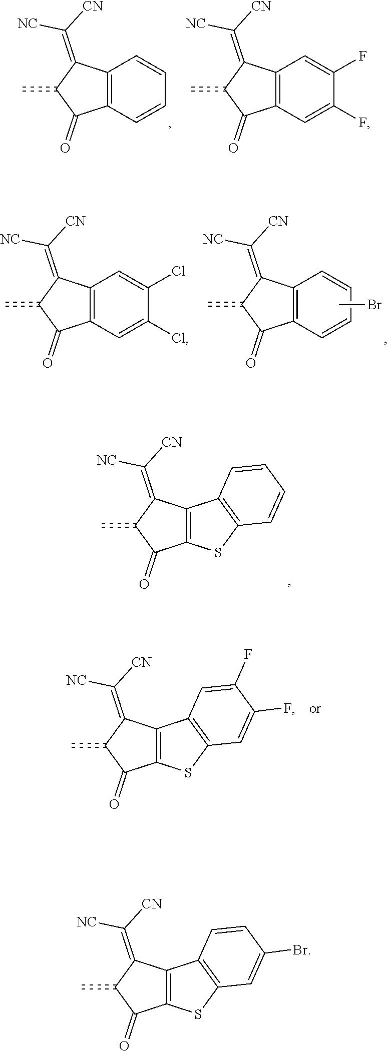

According to some embodiments of the present disclosure, the Buchwald-Hartwig amination reaction is carried out at a temperature of 100° C. to 120° C.

According to some embodiments of the present disclosure, the method of manufacturing the fused ring acceptor material excludes adding any organophosphorus reagent in each step.

According to some embodiments of the present disclosure, a reaction temperature of each step does not exceed 130° C.

According to some embodiments of the present disclosure, the condensation reaction between the compound 4 and the A₁ group-containing acceptor is carried out at a temperature of 30° C. to 50° C.

### BRIEF DESCRIPTION OF THE DRAWINGS

In order to make the above and other objects, features, advantages and embodiments of the present disclosure more clearly understandable, the detailed description of the attached drawings is as follows:

FIG. 1 is a flow chart of a method of manufacturing a fused ring non-fullerene acceptor material according to various embodiments of the present disclosure.

FIG. 2 is a flow chart of a method of manufacturing a compound having dibromothienothiophene according to various embodiments of the present disclosure.

FIG. 3 shows absorption spectra of Example 1 and Comparative Example 1 in a thin film state.

FIG. 4 is a current-voltage (J-V) curve of an organic solar cell prepared in Example 1.

FIG. 5 is an external quantum efficiency (EQE) curve of the organic solar cell prepared in Example 1.

### DETAILED DESCRIPTION

Multiple embodiments of the present disclosure are disclosed below. For the sake of clarity, many practical details will be explained together in the following description. However, it should be understood that these practical details should not be used to limit the present disclosure. That is, in some embodiments of the present disclosure, these practical details are not necessary. In addition, chemical structural formulas shown in the description will be drawn in a simple schematic manner.

In this description, unless the context specifically dictates otherwise, “a” and “the” may mean a single or a plurality. It will be further understood that “comprise”, “include”, “have” and similar terms as used herein indicate described features, regions, integers, steps, operations, elements and/or components, but do not exclude one or more of other features, regions, integers, steps, operations, elements, components, and/or groups thereof described or additionally.

As used in this description, “about”, “approximately” or “substantially about” generally means that an error or a range of a numerical value is within about 20%, preferably within about 10%, and more preferably within about 5%. Unless explicitly stated otherwise, numerical values mentioned in the description are regarded as approximations, that is, errors or ranges expressed by “about”, “approximately” or “substantially about”.

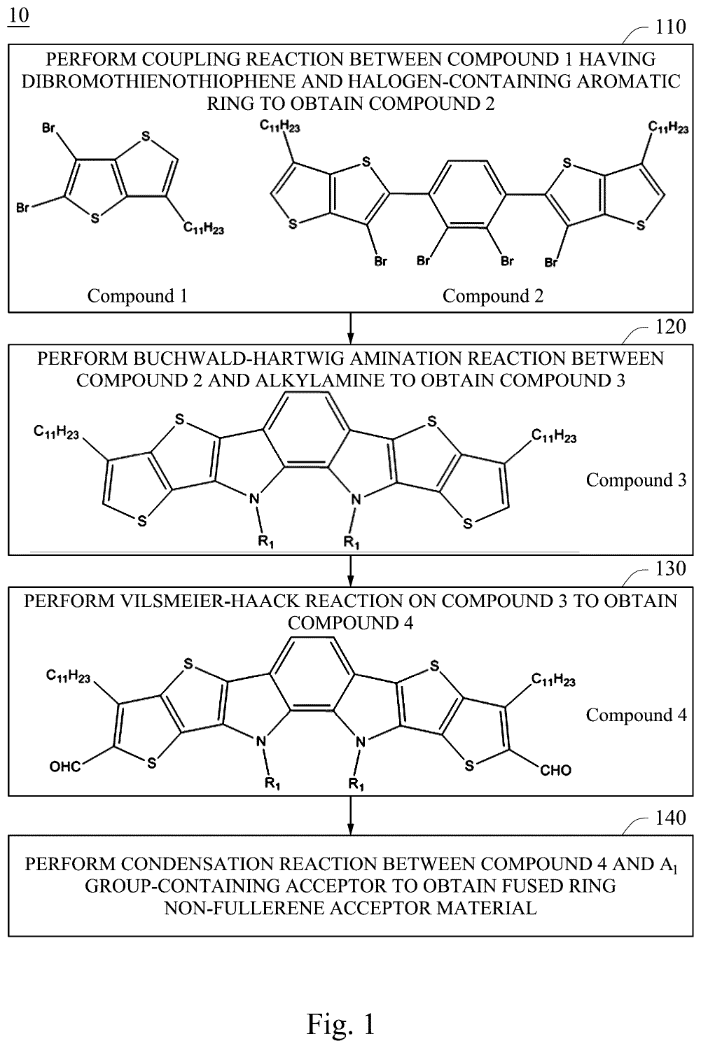

The present disclosure provides a method of manufacturing a fused ring non-fullerene acceptor material with simplified reaction steps and low cost. The fused ring non-fullerene acceptor material obtained by this manufacturing method has a wider selectivity of a central structure, and can also achieve higher photoelectric conversion efficiency when applied to organic solar cells. FIG. 1 is a flow chart of a method 10 of manufacturing a fused ring non-fullerene acceptor material according to various embodiments of the present disclosure. As shown in FIG. 1, the method 10 of manufacturing the fused ring non-fullerene acceptor material includes Step 110, Step 120, Step 130 and Step 140.

Please refer to FIG. 1 first. In Step 110, a coupling reaction is performed between a compound 1 having dibromothienothiophene and a halogen-containing aromatic ring to obtain a compound 2. The compound 1 is

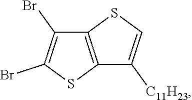

and the compound 2 is

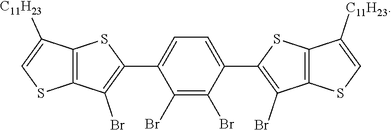

In various embodiments, the halogen-containing aromatic ring may be, for example, 1,4-diiodo-2,3-dibromo benzene,

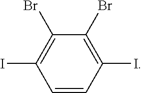

In various embodiments, the coupling reaction in Step 110 is stirred at a temperature of 50° C. to 70° C.

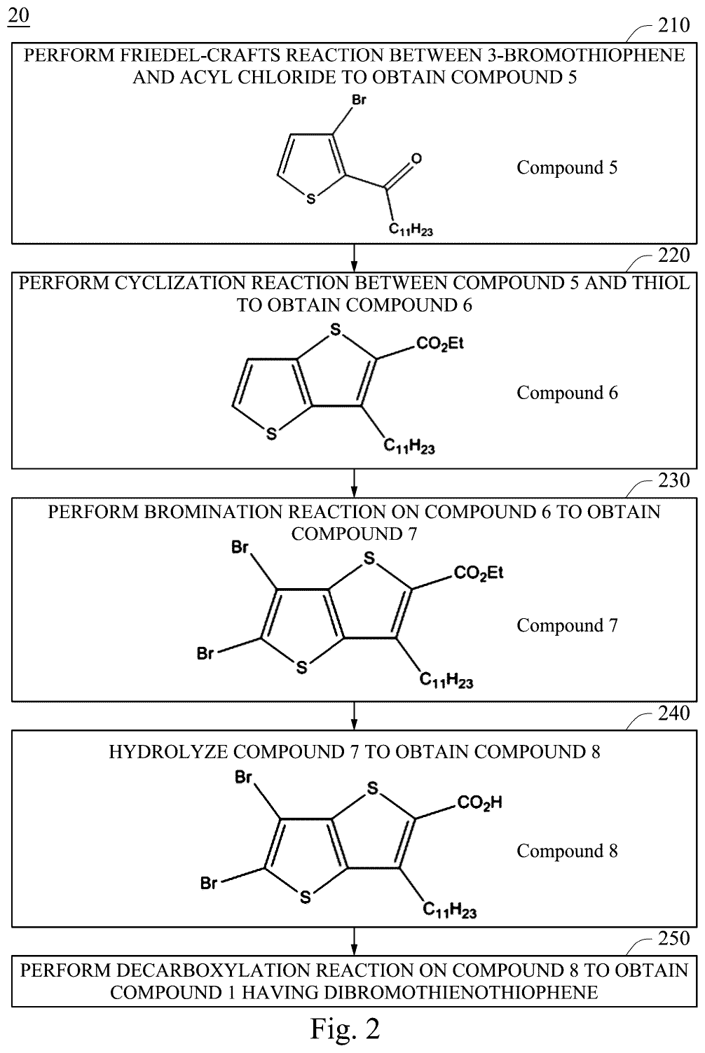

FIG. 2 is a flow chart of a method 20 of manufacturing a compound having dibromothienothiophene according to various embodiments of the present disclosure. As shown in FIG. 2, the method 20 of manufacturing the compound 1 having dibromothienothiophene includes Step 210, Step 220, Step 230, Step 240 and Step 250.

As shown in FIG. 2, in Step 210, a Friedel-crafts reaction is performed between 3-bromothiophen

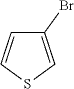

and acyl chloride to obtain a compound 5. The compound 5 is

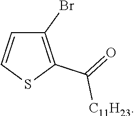

In various embodiments, acyl chloride may be, for example, dodecanoyl chloride. In various embodiments, the Friedel-crafts reaction in Step 210 is performed with mixing at a temperature of 0° C. and returned to room temperature (about 25° C.) with continued stirring.

As shown in FIG. 2, in Step 220, a cyclization reaction is performed between the compound 5 and thiols/mercaptans to obtain a compound 6. The compound 6 is

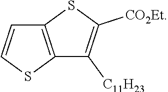

In various embodiments, the thiol may be, for example, ethyl thioglycolate,

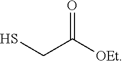

In various embodiments, the cyclization reaction in Step 220 is performed at room temperature (about 25° C.).

As shown in FIG. 2, in Step 230, a bromination reaction is performed on the compound 6 to obtain a compound 7. The compound 7 is

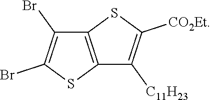

In various embodiments, the bromination reaction is performed between the compound 6 and bromine (Br₂). In various embodiments, the bromination reaction in Step 230 is performed with mixing at room temperature (about 25° C.), followed by heating to about 55° C.-75° C. and stirring.

As shown in FIG. 2, in Step 240, the compound 7 is hydrolyzed to obtain a compound 8. The compound 8 is

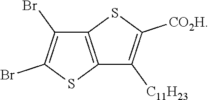

In various embodiments, the hydrolysis reaction in Step 240 is performed at a temperature of 65° C. to 85° C.

As shown in FIG. 2, in Step 250, a decarboxylation reaction is performed on the compound 8 to obtain the compound 1 having dibromothienothiophene. In various embodiments, the decarboxylation reaction in Step 250 is performed at a temperature of 110° C. to 130° C.

Please return to FIG. 1. In step 120, a Buchwald-Hartwig amination reaction is performed between the compound 2 and alkylamine to obtain a compound 3. The compound 3 is

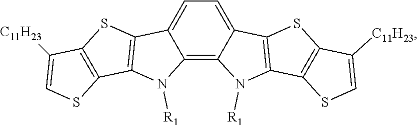

where R₁ may be a C₁-C₂₄ alkyl, a phenyl, a phenyl derivative, a furyl, a furyl derivative, a thienyl, a thienyl derivative, a selenophene or a selenophene derivative. In various embodiments, the alkylamine may be, for example, 2-hexyl decan-1-amine,

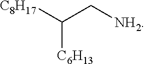

In various embodiments, the Buchwald-Hartwig amination reaction in Step 120 is performed at a temperature of 100° C. to 120° C., such as 105° C., 110° C., or 115° C.

Specifically, in the process of synthesizing the fused ring non-fullerene acceptor material with a structure as shown in formula (I), forming a central structure with a nitrogen bridge (i.e., D^{N}B^{N}D) is a crucial step. As described in the prior art, the current synthesis step of the central structure (D^{N}A₂^{N}D) of the highly efficient organic non-fullerene acceptor material (TPBT-4F) must use the highly toxic organophosphorus reagent (e.g., triethyl phosphite) and the Cadogan reductive cyclization reaction is carried out at the high temperature of 180° C., and the yield (approximately 20% to 30%) is not ideal. However, the present disclosure utilizes a tetrabromo compound to perform the Buchwald-Hartwig amination cyclization reaction under catalysis of palladium metal without the need to add the organophosphorus reagent.

Continuing to refer to FIG. 1, in Step 130, a Vilsmeier-Haack reaction is performed on the compound 3 to obtain a compound 4. The compound 4 is

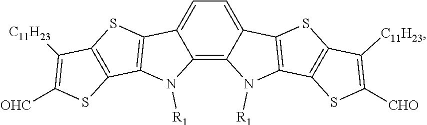

where R₁ may be a C₁-C₂₄ alkyl, a phenyl, a phenyl derivative, a furyl, a furyl derivative, a thienyl, a thienyl derivative, a selenophene or a selenophene derivative. In various embodiments, the Vilsmeier-Haack reaction in Step 130 is performed at a temperature of 65° C. to 85° C.

Continuing to refer to FIG. 1, in Step 140, a condensation reaction is performed between the compound 4 and an A₁ group-containing acceptor to obtain a fused ring non-fullerene acceptor material. Specifically, the A₁ group-containing acceptor may include

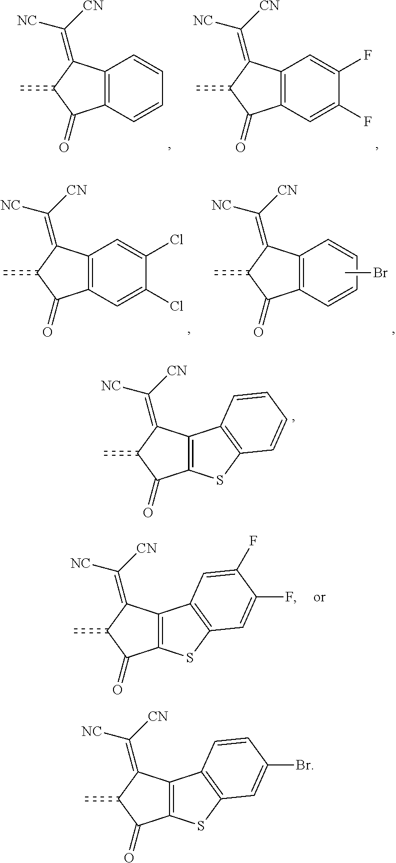

In one embodiment, the A₁ group-containing acceptor may be 2-(5,6-difluoro-3-oxo-2,3-dihydro-1H-inded-1-ylidene)malononitrile,

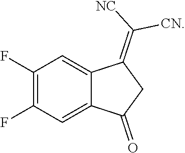 [SMILES: N#CC(C#N)=C1CC(=O)c2cc(F)c(F)cc21]

In various embodiments, the condensation reaction in Step 140 is performed at a temperature of 30° C. to 50° C., such as 35° C., 40° C., or 45° C.

In various embodiments, in the method of manufacturing the fused ring non-fullerene acceptor material of the present disclosure, no organophosphorus reagent is added in each step. In various embodiments, in the method of manufacturing the fused ring non-fullerene acceptor material of the present disclosure, a reaction temperature of each step does not exceed 130° C.

Specifically, the fused ring non-fullerene acceptor material has the structure of the following formula (I):

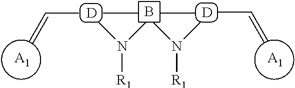

formula (I), where R₁ is a C₁-C₂₄ alkyl, a phenyl, a phenyl derivative, a furyl, a furyl derivative, a thienyl, a thienyl derivative, a selenophene or a selenophene derivative; B is

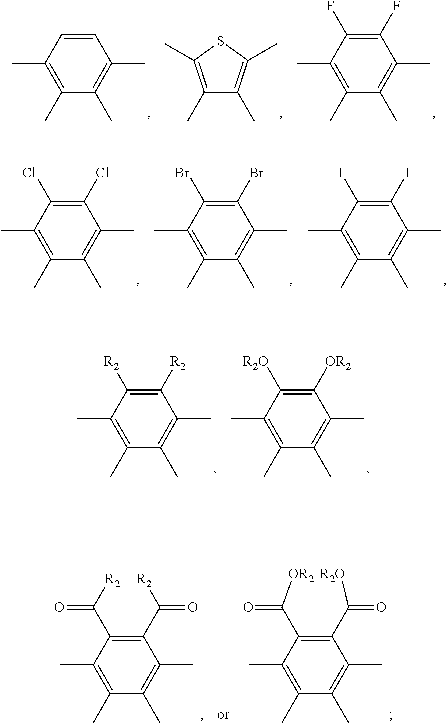

R₂ is a C₁-C₂₄ alkyl, a phenyl, a phenyl derivative, a thienyl or a thienyl derivative; D is

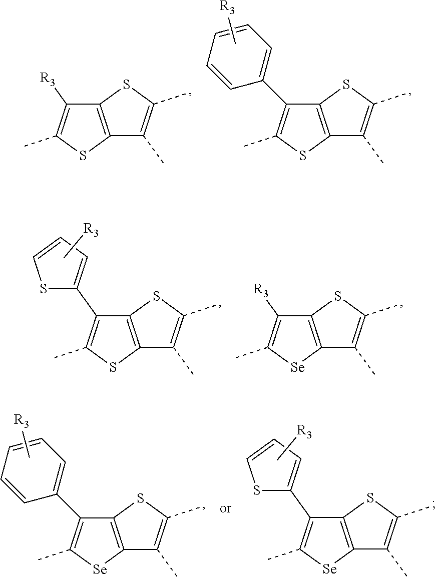

R₃ is a hydrogen atom, a halogen, a C₁-C₂₄ alkyl or a C₁-C₂₄ alkoxy; and A₁ is

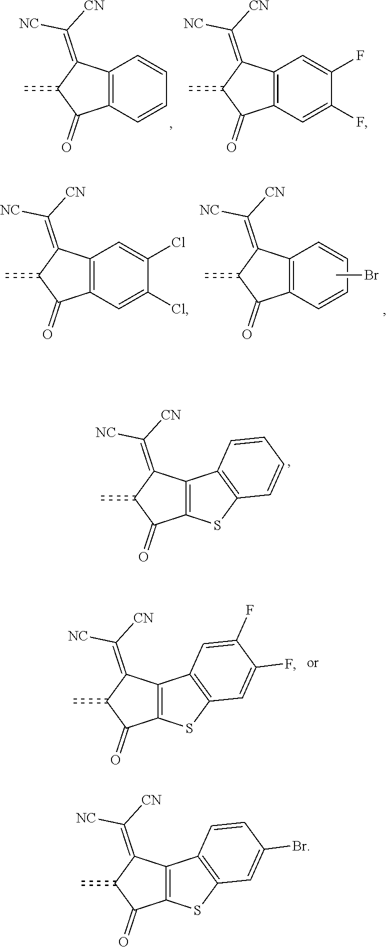

For example, R₁ may be a C₂ alkyl, a C₃ alkyl, a C₄ alkyl, a C₅ alkyl, a C₆ alkyl, a C₇ alkyl, a C₈ alkyl, a C₉ alkyl, a C₁₀ alkyl, a C₁₁ alkyl, a C₁₂ alkyl, a C₁₃ alkyl, a C₁₄ alkyl, a C₁₅ alkyl, a C₁₆ alkyl, a C₁₇ alkyl, a C₁₈ alkyl, a C₁₉ alkyl, a C₂₀ alkyl, a C₂₁ alkyl, a C₂₂ alkyl or a C₂₃ alkyl.

For example, R₂ may be a C₂ alkyl, a C₃ alkyl, a C₄ alkyl, a C₅ alkyl, a C₆ alkyl, a C₇ alkyl, a C₈ alkyl, a C₉ alkyl, a C₁₀ alkyl, a C₁₁ alkyl, a C₁₂ alkyl, a C₁₃ alkyl, a C₁₄ alkyl, a C₁₅ alkyl, a C₁₆ alkyl, a C₁₇ alkyl, a C₁₈ alkyl, a C₁₉ alkyl, a C₂₀ alkyl, a C₂₁ alkyl, a C₂₂ alkyl or a C₂₃ alkyl.

For example, R₃ may be a C₂ alkyl, a C₃ alkyl, a C₄ alkyl, a C₅ alkyl, a C₆ alkyl, a C₇ alkyl, a C₈ alkyl, a C₉ alkyl, a C₁₀ alkyl, a C₁₁ alkyl, a C₁₂ alkyl, a C₁₃ alkyl, a C₁₄ alkyl, a C₁₅ alkyl, a C₁₆ alkyl, a C₁₇ alkyl, a C₁₈ alkyl, a C₁₉ alkyl, a C₂₀ alkyl, a C₂₁ alkyl, a C₂₂ alkyl or a C₂₃ alkyl.

For example, R₃ may be a C₂ alkoxy, a C₃ alkoxy, a C₄ alkoxy, a C₅ alkoxy, a C₆ alkoxy, a C₇ alkoxy, a C₈ alkoxy group, a C₉ alkoxy group, a C₁₀ alkoxy group, a C₁₁ alkoxy group, a C₁₂ alkoxy group, a C₁₃ alkoxy group, a C₁₄ alkoxy group, a C₁₅ alkoxy group, a C₁₆ alkoxy group, a C₁₇ alkoxy group, a C₁₈ alkoxy group, a C₁₉ alkoxy group, a C₂₀ alkoxy group, a C₂₁ alkoxy group, a C₂₂ alkoxy group or a C₂₃ alkoxy group.

Features of the present disclosure will be described in more detail below with reference to experimental examples. Although the following experimental examples are described, the materials used, their amounts and ratios, processing details, processing procedures, etc. may be appropriately changed without exceeding the scope of the present disclosure. Therefore, the present disclosure should not be interpreted restrictively by the experimental examples described below.

### Experimental Example 1: Preparation of Compound 1 Having Dibromothienothiophene

In this experimental example, according to the method 20 of manufacturing the compound 1 having dibromothienothiophene as shown in FIG. 2, a Friedel-crafts reaction was performed between 3-bromothiophene and acyl chloride to obtain a compound 5, please refer to following reaction formula for the preparation process.

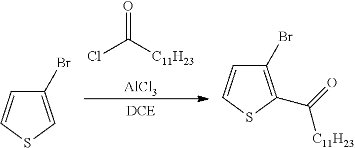

The preparation process will be described in detail below. 3-4 drops of N,N-dimethylformamide (DMF) was added to a solution of dodecanoic acid (10.00 g, 50.0 mmol) in thionyl chloride (20 ml), and a mixture was stirred at reflux for 1 hour. The solvent was removed by distillation to obtain a yellow oil. At 0° C., a mixture of 3-bromothiophene (9.8 g, 60.0 mmol) and dodecanoyl chloride in 1,2-dichloroethane (DCE) (50 ml) was added dropwise to a solution of aluminum chloride (AlCl₃) (7.35 g, 55.0 mmol) in DCE (100 ml). Next, it was returned to room temperature and kept stirring for 3 hours, and the mixture was then poured into ice. A reaction solution was extracted with dichloromethane (DCM) (100 ml×2) and water (200 ml). A collected organic layer was dried over anhydrous magnesium sulfate (MgSO₄). After the solvent was removed under reduced pressure, a residue was purified by flash column on silica gel to obtain a brown viscous oily compound 5 (15.19 g, yield 88%).

Next, a cyclization reaction was performed between the compound 5 and thiol to obtain a compound 6. Please refer to following reaction formula for the preparation process.

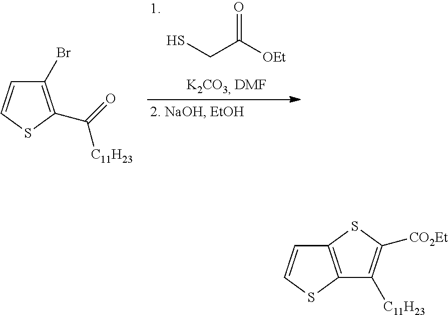

The preparation process will be described in detail below. At room temperature, ethyl thioglycolate (3.5 ml, 27.5 mmol) was slowly added to a solution of the compound 5 (8.65 g, 25.0 mmol) and potassium carbonate (6.90 g, 50.0 mmol) in DMF (50 ml). After stirring at room temperature for 24 hours, another solution containing sodium hydroxide (NaOH) (1.00 g, 25.0 mmol) and ethanol (EtOH) (50 ml) was added to the mixture and stirring was continued at room temperature for 24 hours. A reaction solution was extracted with ethyl acetate (100 ml×4) and water (200 ml). A collected organic layer was dried over anhydrous magnesium sulfate. After the solvent was removed under reduced pressure, a residue was purified by column chromatography on silica gel (hexane/ethyl acetate, v/v, 20/1) to obtain a yellow viscous oily compound 6 (6.87 g, yield 75%).

Next, a bromination reaction was performed on the compound 6 to obtain a compound 7. Please refer to following reaction formula for the preparation process.

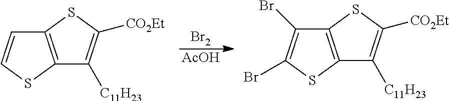

The preparation process will be described in detail below. At room temperature, bromine (1.05 ml, 20.5 mmol) was added dropwise to a solution of the compound 6 (3.67 g, 10 mmol) and acetic acid (12.5 ml). After stirring at room temperature for 2 hours, the mixture was heated to 65° C. and stirred for 16 hours. The mixture was then poured into NaOH solution (1 M, 50 ml) to quench the bromine. A reaction solution was extracted with DCM (50 ml×2) and water (100 ml). A collected organic layer was dried over anhydrous magnesium sulfate. After the solvent was removed under reduced pressure, a residue was recrystallized using DCM/methanol to obtain a white solid compound 7 (3.92 g, yield 75%).

Next, the compound 7 was hydrolyzed to obtain a compound 8. Please refer to following reaction formula for the preparation process.

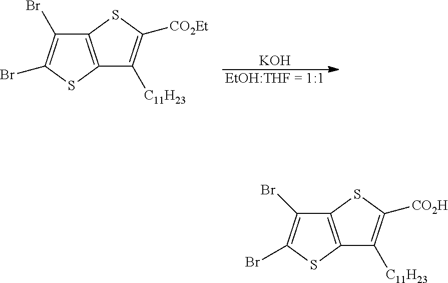

The preparation process will be described in detail below. Potassium hydroxide (KOH) (1.02 g, 18.2 mmol) was added to a solution of the compound 7 (3.41 g, 6.5 mmol) and ethanol/tetrahydrofuran (EtOH/THF) (24 ml, 1/1, v/v). The mixture was stirred at reflux for 12 hours. After cooling to room temperature, the reaction solution was poured into concentrated hydrochloric acid (HCl). An obtained product was filtered. After precipitation, washing with water several times, and vacuum drying, a yellow solid compound 8 (3.00 g, yield 93%) was obtained.

A decarboxylation reaction was performed on the compound 8 to obtain a compound 1 having dibromothienothiophene. Please refer to following reaction formula for the preparation process.

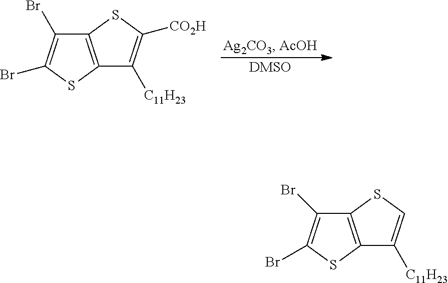

The preparation process will be described in detail below. Acetic acid (17.1 μL, 0.3 mmol) was added to a solution of the compound 8 (2.98 g, 6.0 mmol) and silver carbonate (165.5 mg, 0.6 mmol) in dimethyl sulfoxide (DMSO) (12 ml). The mixture was stirred at 120° C. for 16 hours. The reaction solution was filtered through celite, and a solution was extracted with ethyl acetate (100 ml) and water (50 ml×2). A collected organic layer was dried over anhydrous magnesium sulfate. After the solvent was removed under reduced pressure, a residue was purified by column chromatography on silica gel (hexane) to obtain a colorless oily compound 1 (2.50 g, yield 92%).

### Experimental Example 2: Preparation of Fused Ring Non-Fullerene Acceptor Material

In this experimental example, according to the method 10 of manufacturing the fused ring non-fullerene acceptor material as shown in FIG. 1, a coupling reaction was performed between the previously obtained compound 1 having dibromothienothiophene and a halogen-containing aromatic ring to obtain a compound 2. Please refer to following reaction formula for the preparation process.

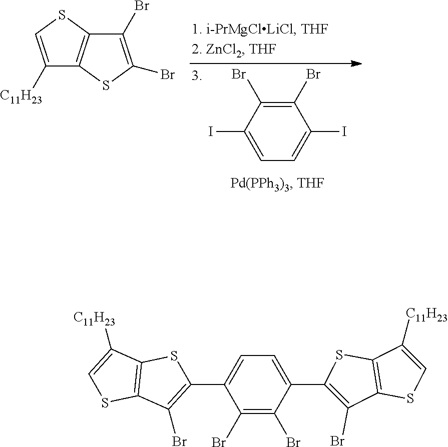

The preparation process will be described in detail below. First, a zinc reagent solution was prepared by the following procedure. At 0° C. and nitrogen, isopropylmagnesium chloride lithium chloride complex solution (1.3 M in THF, 1 ml, 1.30 mmol) was added dropwise to a solution of the compound 1 (588.0 mg, 1.30 mmol) in anhydrous THE (2.5 ml). Next, after stirring at 0° C. for 1 hour, another solution of zinc chloride (204.4 mg, 1.50 mmol) in anhydrous THE (1 ml) was added, and continue stirring for 1 hour to obtain the zinc reagent solution. At 0° C., 1,4-dibromo-2,3-diiodobenzene (243.9 mg, 0.50 mmol) and bis(triphenylphosphine)palladium(II) dichloride (PdCl₂(PPh₃)₂) (35.1 mg, 0.05 mmol) in anhydrous THE (2 ml) was slowly added to the zinc reagent solution. The mixture was stirred at reflux for 16 hours. After cooling to room temperature, the reaction solution was quenched with water and extracted with DCM (25 ml×2) and water (25 ml). A collected organic layer was dried over anhydrous magnesium sulfate. After the solvent was removed under reduced pressure, a residue was purified by column chromatography on silica gel (hexane) to obtain a light yellow oily compound 2 (266.1 mg, yield 70%).

Next, a Buchwald-Hartwig amination reaction was performed between the compound 2 and alkylamine to obtain a compound 3. Please refer to following reaction formula for the preparation process.

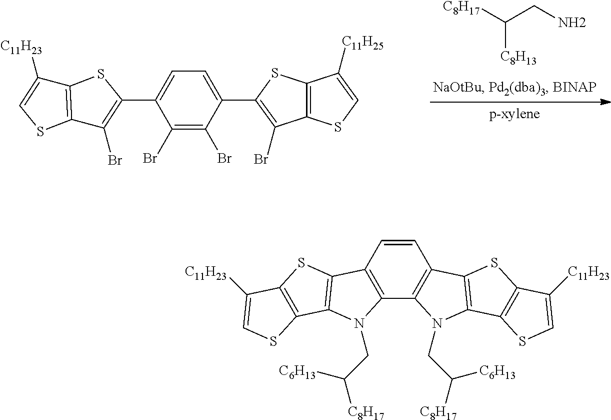

The preparation process will be described in detail below. 2-hexyldecan-1-amine (434.6 mg, 1.80 mmol) was added to a solution of the compound 2 (293.6 mg, 0.30 mmol), sodium tert-butoxide (259.5 mg, 2.70 mmol), racemic-2,2′-Bis(diphenylphosphino)-1,1′-binaphthyl (BINAP) (149.4 mg, 0.24 mmol) and Pd₂(dba)₃ (54.9 mg, 0.06 mmol) in degassed p-xylene (3.6 ml). The mixture was stirred at 110° C. for 20 hours. The reaction solution was filtered through celite and extracted with DCM (25 ml×2) and water (50 ml). A collected organic layer was dried over anhydrous magnesium sulfate. After the solvent was removed under reduced pressure, a residue was purified by column chromatography on silica gel (hexane) to obtain a yellow oily compound 3 (112.7 mg, yield 33%).

Next, a Vilsmeier-Haack reaction was performed on the compound 3 to obtain a compound 4. Please refer to following reaction formula for the preparation process.

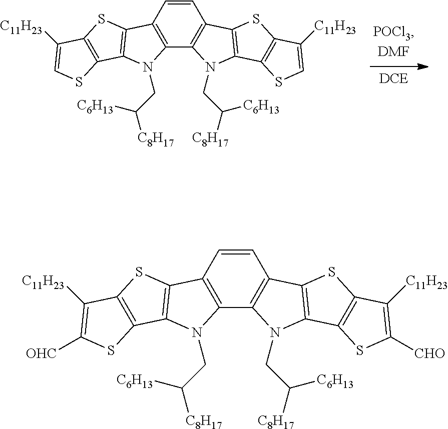

The preparation process will be described in detail below. Phosphoryl chloride (POCl₃) (82.0 μl, 0.90 mmol) was added to DMF (1 ml) and stirred at 0° C. for 30 minutes to obtain a first mixture. The first mixture was added dropwise to a solution of the compound 3 (100.4 mg, 0.09 mmol) in DCE (10.5 ml) and stirred at 85° C. for 16 hours. It was poured into water and stirred for 30 minutes. A reaction solution was extracted with DCM (25 ml×2) and water (50 ml). A collected organic layer was dried over anhydrous magnesium sulfate. After removing the solvent under reduced pressure, a residue was purified by column chromatography on silica gel (hexane/DCM, v/v, 3/2) to obtain a viscous fluorescent yellow oily compound 4 (77.1 mg, yield 72%).

Next, a condensation reaction was performed between the compound 4 and an A₁ group-containing acceptor to obtain the fused ring non-fullerene acceptor material of Example 1 of the present disclosure. Please refer to following reaction formula for the preparation process.

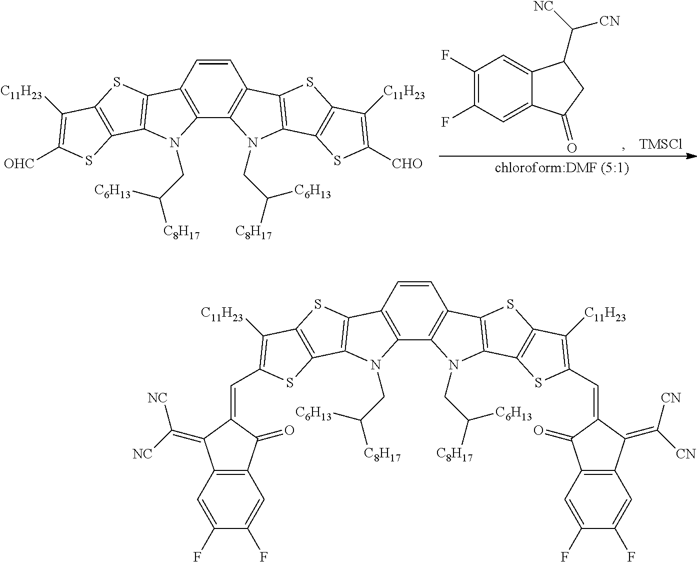

The preparation process will be described in detail below. Trimethylsilyl chloride (1.5 ml, 11.8 mmol) was added dropwise to a solution of the compound 4 (77.1 mg, 0.06 mmol) and 2-(5,6-difluoro-3-oxo-2,3-dihydro-1H-inden-1-ylidene)malononitrile (FIC) (59.5 mg, 0.24 mmol) in chloroform/DMF (7.2 ml, 5/1, v/v). The mixture was stirred at 40° C. for 16 hours. A reaction solution was extracted with DCM (50 ml×2) and water (75 ml). A collected organic layer was dried over anhydrous magnesium sulfate. After removing the solvent under reduced pressure, a residue was purified by column chromatography on silica gel (hexane/DCM, v/v, 1/1) to obtain a dark blue solid fused ring non-fullerene acceptor material, which was Example 1 (hereinafter referred to as c-BDPTT-FIC) (88.3 mg, yield 91%).

Chemical shifts (6) (unit: ppm) of nuclear magnetic resonance (NMR) hydrogen spectrum (¹H-NMR (400 MHz, CDCl₃)) of Example 1 (c-BDPTT-FIC): 9.14 (s, 2H), 8.56 (dd, J=6.8, 3.1 Hz, 2H), 7.69 (t, J=7.5 Hz, 2H), 7.59 (s, 2H), 4.67 (d, J=7.4 Hz, 4H), 3.20 (t, J=7.3 Hz, 4H), 2.03 (br. s, 2H), 1.89-1.81 (m, 4H), 1.54-1.47 (m, 4H), 1.37-1.26 (m, 34H), 1.21-0.85 (m, 56H) 0.81-0.69 (m, 18H). ¹³C NMR (100 MHz, CDCl₃): δ 186.14, 159.07, 155.73, 154.02, 153.15, 144.07, 138.75, 136.91, 136.71, 136.65, 135.42, 134.59, 133.50, 133.29, 131.28, 123.27, 119.53, 115.18, 114.99, 114.81, 112.43, 68.24, 55.09, 38.94, 31.98, 29.86, 29.53, 29.46, 29.34, 25.53, 22.78, 22.73, 22.63, 14.22, 14.17.

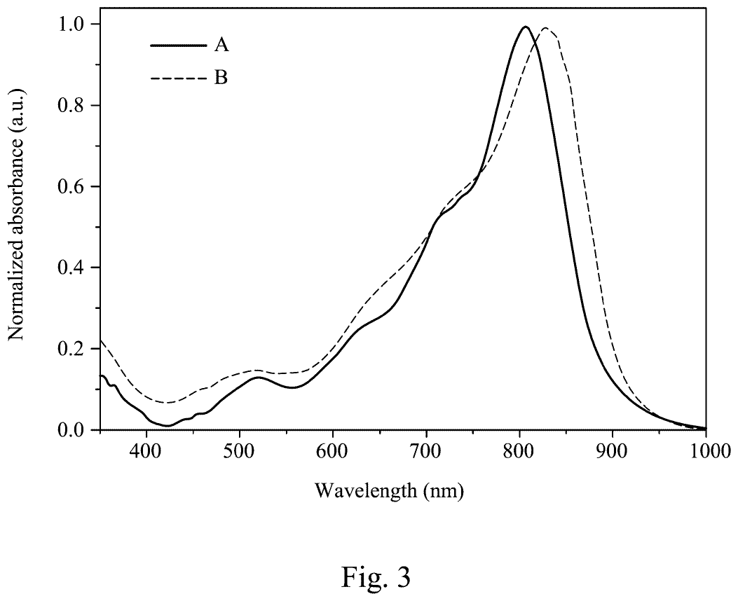

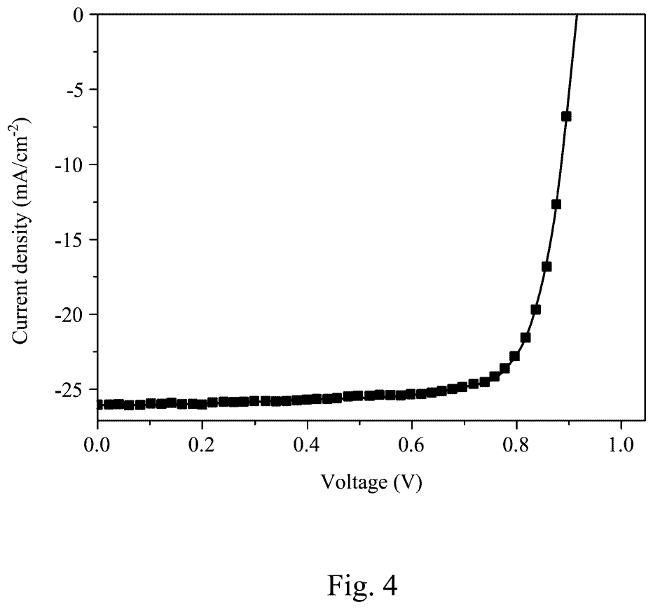

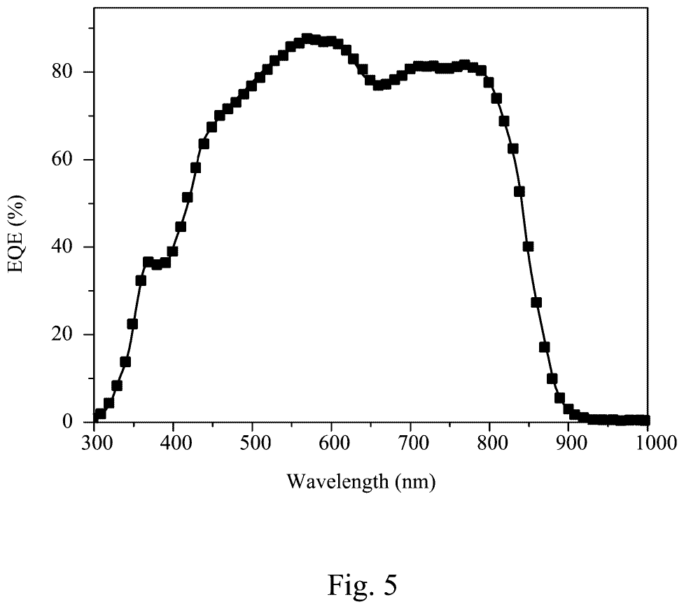

FIG. 3 shows absorption spectra of Example 1 and Comparative Example 1 in a thin film state, in which a curve A represents Example 1, and a curve B represents Comparative Example 1. FIG. 4 is a current-voltage (J-V) curve of an organic solar cell prepared in Example 1. FIG. 5 is an external quantum efficiency (EQE) curve of the organic solar cell prepared in Example 1. Specifically, Example 1 was the acceptor material c-BDPTT-FIC obtained by the above-mentioned manufacturing method, and Comparative Example 1 was a commercial acceptor material Y6. Specifically, commercial PM6 was used as a donor material, and was used as an active layer of an organic solar cell in combination with Example 1 (c-BDPTT-FIC) and Comparative Example 1 (Y6). Organic solar cells were made based on a structure of ITO/ZnO/active layer/MnO₃/Ag. Performance parameters of the organic solar cells of Example 1 and Comparative Example 1 at different ratios are listed in Table 1 below, and are plotted as shown in FIGS. 3, 4 and 5.

TABLE 1

| | | V_{oc} | J_{sc} | FF | PCE |
|---|---|---|---|---|---|
| Active layer | Ratio | (V) | (mA/cm²) | (%) | (%) |
| PM6:Example 1 | 1:1.2 | 0.92 | 25.98 | 76.89 | 18.32 |
| PM6:Comparative Example 1 | 1:1.2 | 0.84 | 25.20 | 76.10 | 15.70 |

As can be seen from Table 1 and FIGS. 3 to 5, compared with Comparative Example 1, Example 1 of the present disclosure had a higher open circuit voltage (V_{oc}) in the application of organic solar cell and a similar short circuit current (J_{sc}) and a fill factor (FF), and power conversion efficiency (PCE) was also higher.

In summary, the present disclosure provides the method of manufacturing the fused ring non-fullerene acceptor material with the simplified reaction steps and reaction times and low cost, and the yield is as high as about 30% to 50%. The fused ring non-fullerene acceptor material obtained by this manufacturing method has the wider selectivity of the central structure, and can also achieve the higher photoelectric conversion efficiency when applied to the organic solar cells.

Although the present disclosure has been disclosed as above in embodiments, the above is only preferred embodiments of the present disclosure and is not intended to limit the present disclosure. It is to be understood that those skilled in the art can make various equivalent changes and modifications without departing from the spirit and scope of the present disclosure, which should all fall within the scope of the present disclosure. Therefore, the protection scope of the present disclosure is subject to the definition of the scope of appended claims.

What is claimed is:

1. A fused ring acceptor material, comprising a structure of following formula (I): 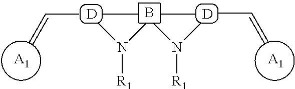
formula (I), where
R₁ is a C₁-C₂₄ alkyl, a phenyl, a phenyl derivative, a furyl, a furyl derivative, a thienyl, a thienyl derivative, a selenophene or a selenophene derivative; 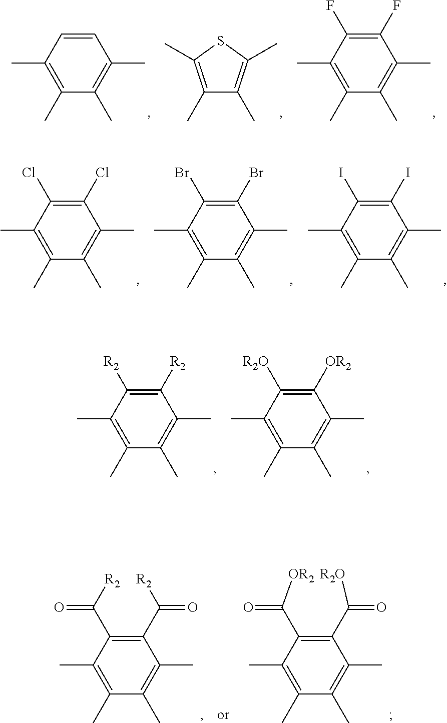
R₂ is a C₁-C₂₄ alkyl, a phenyl, a phenyl derivative, a thienyl or a thienyl derivative;
D is 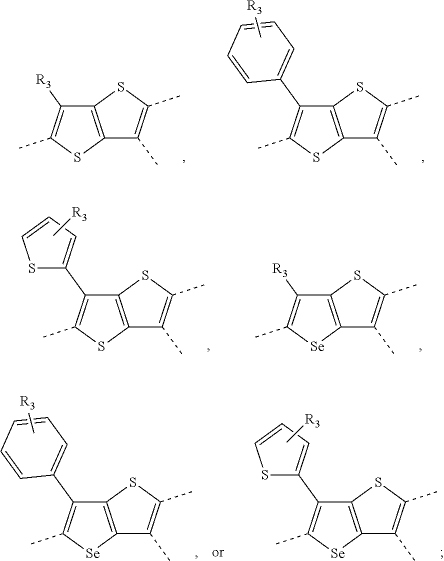
R₃ is a hydrogen atom, a halogen, a C₁-C₂₄ alkyl or a C₁-C₂₄ alkoxy; and
A₁ is

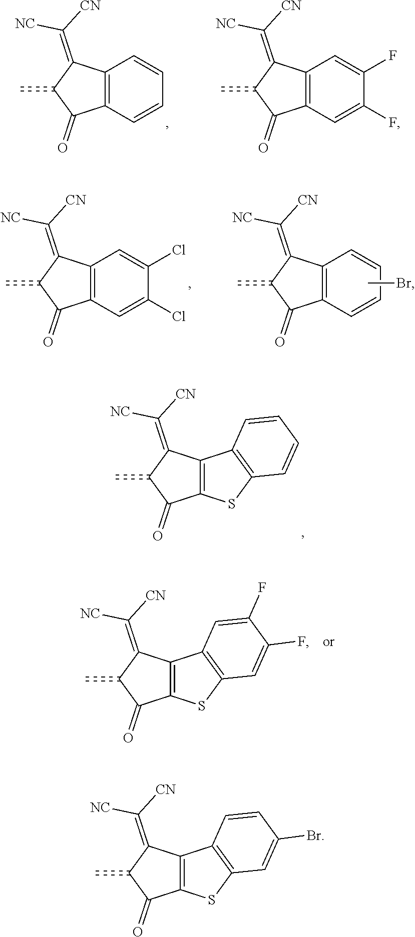

2. The fused ring acceptor material of claim 1, wherein when R₂ is the phenyl derivative, the phenyl derivative comprises at least one group of a C₁-C₂₄ alkyl, a C₁-C₂₄ alkoxy, a halogen or a combination thereof.

3. The fused ring acceptor material of claim 1, wherein when R₂ is the thienyl derivative, the thienyl derivative comprises at least one group of a C₁-C₂₄ alkyl, a C₁-C₂₄ alkoxy, a halogen or a combination thereof.

4. The fused ring acceptor material of claim 1, wherein the fused ring acceptor material is,

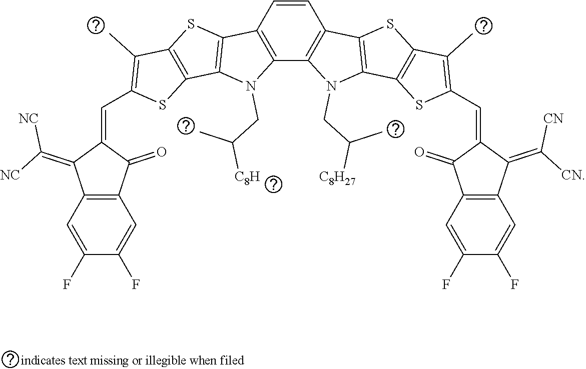

5. A method of manufacturing a fused ring acceptor material, comprising:
performing a coupling reaction between a compound 1 having dibromothienothiophene and a halogen-containing aromatic ring to obtain a compound 2; 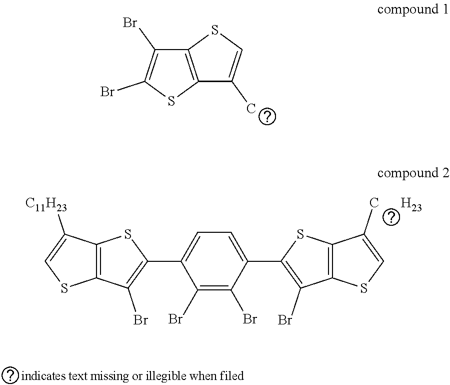
performing a Buchwald-Hartwig amination reaction between the compound 2 and alkylamine to obtain a compound 3; 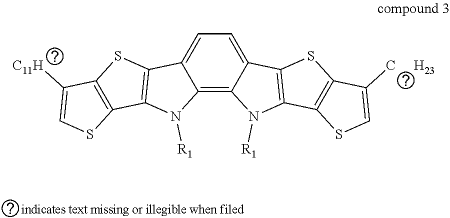
performing a Vilsmeier-Haack reaction on the compound 3 to obtain a compound 4; and 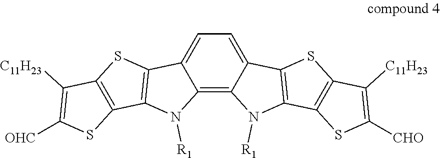
performing a condensation reaction between the compound 4 and an A₁ group-containing acceptor to obtain a fused ring non-fullerene acceptor material, where
the R₁ is a C₁-C₂₄ alkyl, a phenyl, a phenyl derivative, a furyl, a furyl derivative, a thienyl, a thienyl derivative, a selenophene or a selenophene derivative; and
the A₁ group-containing acceptor comprises

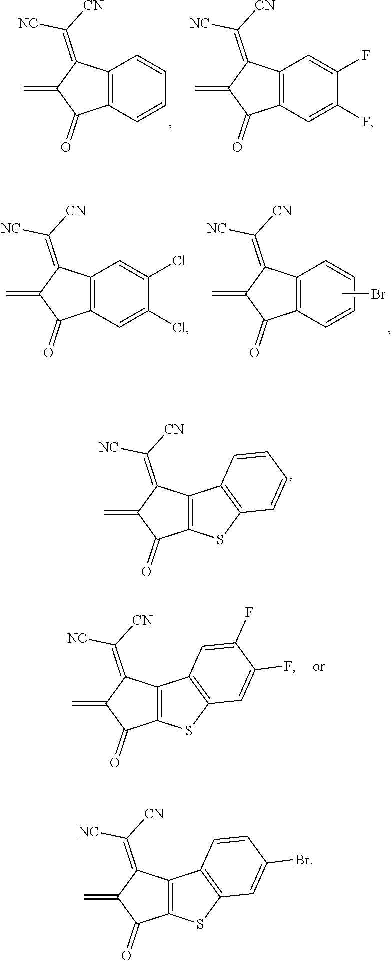

6. The method of manufacturing the fused ring acceptor material of claim 5, wherein forming the compound 1 having dibromothienothiophene comprises:
performing a Friedel-crafts reaction between 3-bromothiophene and acyl chloride to obtain a compound 5; 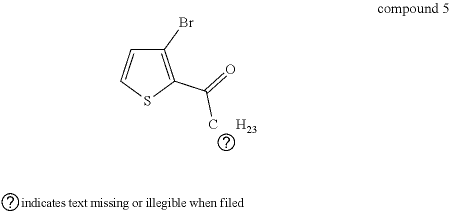
performing a cyclization reaction between the compound 5 and thiol to obtain a compound 6; 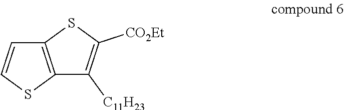
performing a bromination reaction on the compound 6 to obtain a compound 7; 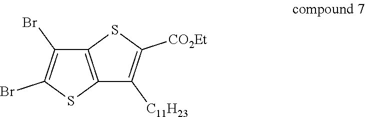
hydrolyzing the compound 7 to obtain a compound 8; and 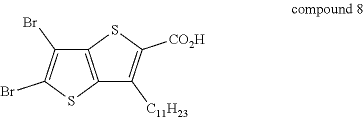
performing a decarboxylation reaction on the compound 8 to obtain the compound 1 having dibromothienothiophene.

7. The method of manufacturing the fused ring acceptor material of claim 5, wherein the fused ring non-fullerene acceptor material has a structure of following formula (I): 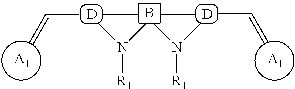
formula (I), where
R₁ is a C₁-C₂₄ alkyl, a phenyl, a phenyl derivative, a furyl, a furyl derivative, a thienyl, a thienyl derivative, a selenophene or a selenophene derivative;
B is 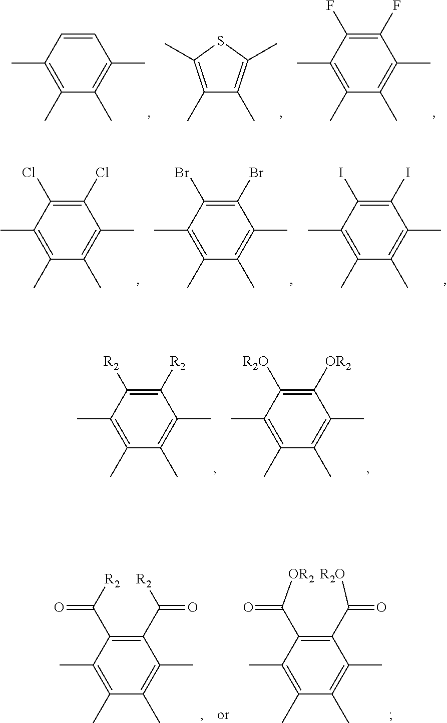
R₂ is a C₁-C₂₄ alkyl, a phenyl, a phenyl derivative, a thienyl or a thienyl derivative;
D is 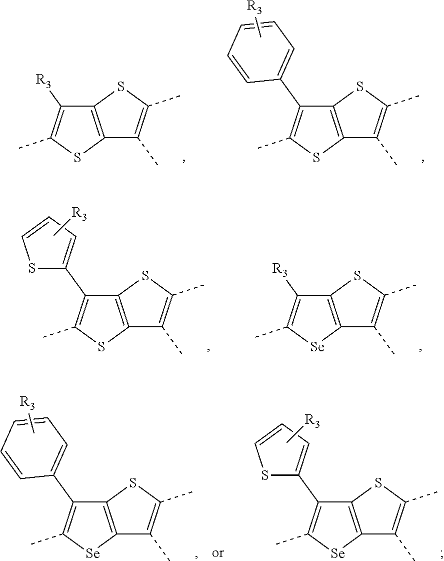
R₃ is a hydrogen atom, a halogen, a C₁-C₂₄ alkyl or a C₁-C₂₄ alkoxy; and
A₁ is

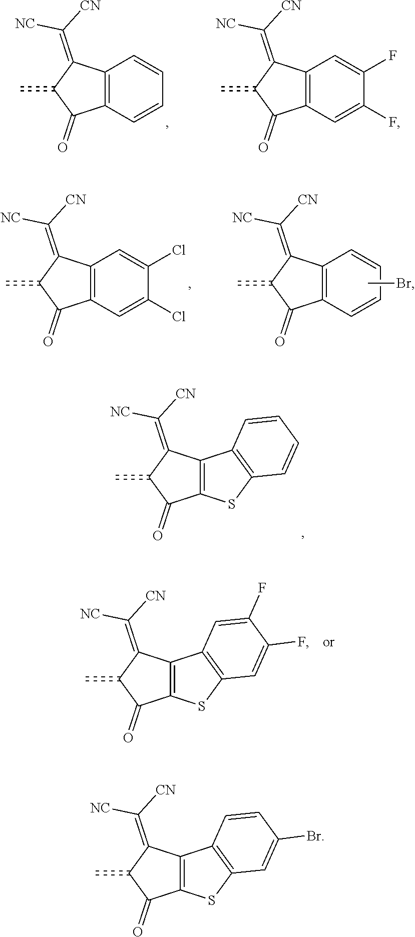

8. The method of manufacturing the fused ring acceptor material of claim 7, wherein when R₂ is the phenyl derivative, the phenyl derivative comprises at least one group of a C₁-C₂₄ alkyl, a C₁-C₂₄ alkoxy, a halogen or a combination thereof.

9. The method of manufacturing the fused ring acceptor material of claim 7, wherein when R₂ is the thienyl derivative, the thienyl derivative comprises at least one group of a C₁-C₂₄ alkyl, a C₁-C₂₄ alkoxy, a halogen or a combination thereof.

10. The method of manufacturing the fused ring acceptor material of claim 7, wherein the fused ring acceptor material is

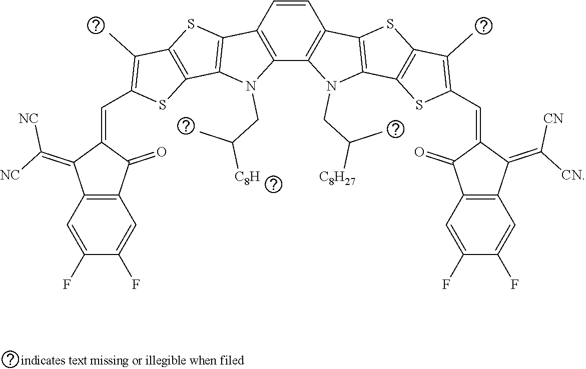

11. The method of manufacturing the fused ring acceptor material of claim 5, wherein the Buchwald-Hartwig amination reaction is carried out at a temperature of 100° C. to 120° C.

12. The method of manufacturing the fused ring acceptor material of claim 5, further comprising not adding any organophosphorus reagent in each step.

13. The method of manufacturing the fused ring acceptor material of claim 5, wherein a reaction temperature of each step does not exceed 130° C.

14. The method of manufacturing the fused ring acceptor material of claim 5, wherein the condensation reaction between the compound 4 and the A₁ group-containing acceptor is carried out at a temperature of 30° C. to 50° C.

15. The method of manufacturing the fused ring acceptor material of claim 5, wherein a yield of the method of manufacturing the fused ring acceptor material is about 30% to about 50%.
